(19) United States
(12) Patent Application Publication
(10) Pub. No.: US 2007/0255208 A1
(43) Pub. Date: Nov. 1, 2007

(54) Percutaneous dilation apparatus

(76) Inventors: Donald J. McMichael, South Jordan, UT (US); John K. Bacon, Provo, UT (US); John A. Rotella, Marietta, GA (US); Kristy Peterson, Santa Rosa, CA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/413,518

(22) Filed: Apr. 28, 2006

(51) Int. Cl. A61M 29/00

(52) U.S. Cl. 604/104

(57) ABSTRACT

A percutaneous dilation apparatus is shown. The apparatus is useful for forming and enlarging percutaneous penetrations to a variety of target locations within a patient's body for multiple purposes. The apparatus includes an at least one dilator segment, a dilator probe, and a mechanism for moving each dilator segment along the dilator probe so as to place it within the penetration thereby dilating the penetration.

### BACKGROUND OF THE INVENTION

The present invention relates generally to a medical device for facilitating the percutaneous access of a body lumen and, more particularly, it relates to the construction and use of a dilator tool or dilation apparatus which enables the sequential radial dilation of a tissue opening to create larger diameter working channels into the body lumen.

Modern medicine frequently requires percutaneous access to hollow body organs, tissue, cavities, and the like. In the case of “least or minimally invasive” surgical procedures, such access is usually provided by inserting a suitable cannula, instrument, tube, or the like, through a small access hole. The initial access is usually created by piercing the skin and any intermediate body structures with a needle or trocar. The initial puncture, however, is usually very small so that the needle or trocar can achieve the desired penetration without excessive damage to tissue. It is therefore necessary for the initial access hole to be subsequently enlarged to provide a working channel having a sufficient diameter to permit performance of the desired medical procedure.

One common technique for achieving such enlargement relies on successively introducing one or more dilating rods having increasingly larger diameters through the puncture hole and into the body organ, tissue, or cavity. When a flexible guide wire has been introduced through the initial needle or cannula puncture, this protocol is referred to as the Seldinger technique.

While this technique is reasonably effective for placement of relatively small devices, e.g., catheters to about 6 French [1 French (F) is equal to 0.079 inch diameter], larger dilations require increasing numbers of dilator exchanges and can be extremely time consuming. Moreover, the body structures that are being penetrated frequently comprise relatively flaccid membranes or walls so that penetration with larger dilators may cause fascial detachment, i.e., the invagination and separation of the membrane or wall from surrounding tissue structures. Such problems may be exacerbated when the organ, tissue, or cavity being penetrated is diseased so that the membranes or walls are thickened or toughened and resistant to penetration by the dilator which axially engages the tissue.

One approach for preventing fascial detachment of the internal body organ or structure during the dilation process involves the use of separate anchoring instruments which are placed around the site of penetration and dilation. The technique, developed by Dr. Cope, relies on the placement of multiple separate anchors or toggles peripherally about the site of the primary puncture in order to more strongly attach the body organ to its surrounding fascia. The anchors are attached to lengths of suture which extend through the tracks defined by the separate punctures. The sutures are tensioned in order to hold the wall of the hollow organ against the fascia and subsequently secured outside the body. While this approach is generally successful, it requires a separate puncture for each anchor and the subsequent suturing of each anchor in place. The technique is therefore relatively time consuming, costly, and potentially subjects the patient to greater discomfort.

An additional problem with the use of successively larger dilators, either with or without use of an anchoring technique, is the leakage of body fluids and substances through the penetration which is being enlarged. While such leakage will be inhibited while each successive dilator is in place, removal of the dilator will allow the fluids from the organ, tissue or cavity being penetrated to contaminate other body structures on the puncture track. For example, percutaneous access to the gallbladder is normally achieved transhepatically since the gallbladder is partially attached to the liver. Transperitoneal access proceeds through an unattached wall of the gallbladder and increases the likelihood of bile leakage into the peritoneal. While transperitoneal access might otherwise be preferred for a number of reasons, e.g., it avoids potential damage to the liver, it is contraindicated by the difficulty in penetrating the unattached wall of the gallbladder and the greater risk of bile leakage associated with conventional dilation techniques.

For these reasons, it would be desirable to provide improved methods and apparatus for forming and enlarging percutaneous penetrations into hollow body organs, tissues, and cavities. The apparatus and methods should be suitable for enlarging percutaneous access penetrations to virtually any diameter, including very large diameters on the order of 20 F, 24 F, and larger while reducing the risk of invagination and fascial detachment of the organ, tissue, or cavity which is being penetrated. The methods should minimize any additional time and complexity required for performing an associated interventional procedure, and in particular, should avoid the need to make secondary penetrations in order to secure the body organ, tissue, or cavity to surrounding fascia. The methods should further avoid complexity and will preferably reduce the number of dilation mechanisms required to achieve a desired enlargement. The method should also lessen the patient discomfort associated with the procedure and should be compatible with virtually any type of interventional procedure which requires the formation of a percutaneous penetration for access to the body organ, tissue, or cavity.

### SUMMARY OF THE INVENTION

In response to the foregoing problems and difficulties encountered by those of skill in the art, the present invention is directed toward a percutaneous dilation apparatus. In one aspect of the invention, the percutaneous dilation apparatus may have a dilator probe, at least one dilator segment, and a plunger. The dilator probe is for entering and enlarging a percutaneous penetration into a target location within a patient's body. The dilator probe may have a handle at a proximal end, a tapered distal end, and defines a length therebetween. The plunger may also have a handle at a proximal end, a distal end, a catch proximal to the distal end, and a length defined between the proximal and distal ends. The plunger slidably engages the dilator probe. The dilator segment or plurality of such segments would have a tapered distal end and be disposed in an initial configuration concentrically about an exterior of the dilator probe and a portion of the plunger. Upon withdrawal of the plunger from the dilator segment and subsequent application of force to the dilator segment by the plunger, the catch moves the dilator segment along the length of the dilator probe to a final position proximate the distal end of the dilator probe. The tapered distal end of the dilator segment enters and enlarges the percutaneous penetration an amount greater than the dilator probe alone. Many embodiments contain a plurality of such concentrically nested dilator segments. The dilator probe and plunger may be arranged to form a central lumen through a longitudinal central axis of the combination to enable the insertion of a penetration device.

In other embodiments, the apparatus may contain a plurality of cylindrical dilator segments graduated in size from smaller to larger, and concentrically arranged about the dilator probe and the next smaller dilator segment. Each segment would be engaged sequentially by the catch and moved toward the distal end to enable enlargement of the penetration.

In yet another embodiment of the device, a percutaneous dilation apparatus may have a movable dilator segment having a tapered distal end adapted to enter and expand a percutaneous penetration in a target location within a patient's body. A dilator probe having a tapered distal end for initial entry and initial enlargement of the percutaneous penetration is provided as well. The dilator probe would be disposed internally and concentrically within the movable dilator segment and would have a mechanism for releasably engaging the movable dilator segment and moving it from an initial position to a deployed position within the percutaneous penetration.

In still another embodiment, a percutaneous dilation apparatus may have a plurality of dilator segments, each diametrically graduated and concentrically nested one within the other. A dilator probe would be provided for initial entry and enlargement of a percutaneous penetration in a target location within a patient's body. The dilator probe would have a mechanism for releasably engaging each movable dilator segment sequentially from smallest to largest and moving each movable dilator segment in turn along the dilator probe and into the penetration thereby dilating the penetration with the insertion of each dilator segment.

Other objects, advantages and applications of the present invention will be made clear by the following detailed description of a preferred embodiment of the invention and the accompanying drawings wherein reference numerals refer to like or equivalent structures.

### BRIEF DESCRIPTION OF THE DRAWINGS

FIG. 1 is a perspective view of one embodiment of the present invention shown as a complete assembly;

FIG. 2 is a perspective view of one component of the FIG. 1 assembly;

FIG. 3 is a perspective view of another component of the FIG. 1 assembly;

FIG. 4 is a perspective view of the FIGS. 2 and 3 components assembled into a subassembly;

FIG. 5 is a cross-section through line A-A of the FIG. 4 subassembly;

FIG. 6 is a cutaway view of a third component of the FIG. 1 assembly; and

FIGS. 7-9 are cutaway views of the FIG. 1 assembly depicting various stages of engagement.

### DETAILED DESCRIPTION

The present invention is useful for forming and enlarging percutaneous penetrations to a variety of target locations within a patient's body for a multiplicity of purposes. The initial penetration will be very small, usually being below about 7 F, more usually being below about 3 F, and frequently being below about 20 GA (gauge; 0.035 in). The penetration will subsequently be enlarged to a desired final size, usually having a final diameter in the range from about 10 French (F) to about 30 F, typically being from about 12 F to 28 F, and usually being from about 14 F to 24 F, with the present invention being particularly useful for the formation of larger diameter penetrations.

The purpose of the penetration may be for drainage, intraorgan drug administration, perfusion, aspiration, or the like, but will usually be for the introduction of a relatively large surgical instrument or working catheter, such as those intended for least invasive surgical procedures. Such procedures include laparoscopy, balloon dilation of ducts, placement of stents, urological and biliary stone removal, ostomy procedures such as colonoscopy, tracheostomy, and the like. Another common purpose for the penetration may be for feeding directly to the gastrointestinal tract such as via a jejunostomy or gastrostomy. Target locations for the percutaneous penetrations will usually be the interior of a hollow body organ or body cavity, such as the gallbladder, stomach, urinary bladder, uterus, kidney, portions of the lung and trachea, rectum, the peritoneum, and the like. The target locations may also be situated within solid tissue as well as solid organs, such as a solid tumor or abscess. Depending on the location which is being accessed, the length and flexibility of the apparatus of the present invention may vary significantly.

A percutaneous dilation apparatus or tool according to the present invention includes an elongate dilator probe, a plunger, at least one dilator segment, and optionally a splittable sheath. The dilator probe and plunger are slidably movable with respect to one another, and together define an axial lumen which defines a path through a central axis of the percutaneous dilation apparatus. The apparatus will have proximal and distal ends, and may have a generally flexible or rigid structure, depending on the particular application. Rigid or semi-rigid components will generally be employed when the target organ may be approached along a substantially straight path, while a more flexible apparatus will be employed when the access route is more tortuous.

The length of the apparatus will vary, with shorter embodiments typically having a length in the range from about 7 cm to 12 cm and being suitable for accessing target locations which are near the surface of the skin, such as the stomach or trachea.

Longer embodiments will have a length in the range from about 15 cm to 25 cm and will be suitable for accessing more remote target locations, such as the kidney. Even longer embodiments having lengths in the range from about 30 cm to 50 cm, or longer, may be employed for accessing the most remote ducts and body locations.

A penetration device having a sharpened tip will optionally be provided in conjunction with the apparatus for puncturing the skin and underlying tissue, organs, and the like, as the apparatus is percutaneously advanced toward its target location. Conveniently, the axial lumen formed by the dilator probe and plunger provides a passage for the introduction of the penetration device in the form of a needle, stylet, or trocar. The penetration device is placed into the axial lumen so that the sharpened tip is exposed at the distal end of the apparatus. The apparatus may then be percutaneously advanced to the target location and the penetration device removed prior to radial expansion of the stoma, as described hereinafter.

A sharpened tip or other means for puncturing the skin will be necessary when no previous needle puncture would have been made. The present invention, however, is useful in cases where conventional techniques and apparatus are used to form an initial, relatively small diameter, puncture. Typically, the puncture will be made using a very small needle, and it will be possible in some cases to introduce the dilation tube of the present invention (without a sharpened tip) directly into the initial puncture track. More commonly, the initial puncture track will be subsequently enlarged to an intermediate diameter using conventional techniques and apparatus, such as the Seldinger technique combined with very small axial dilation. The dilated intermediate diameter will typically be in the range from about 3 F to 8 F, more typically being in the range from about 5 F to 7 F. The apparatus of the present invention may then be introduced into the partially dilated penetration, typically over a flexible guide wire or other member which has been left in place to maintain the track. The penetration may then be enlarged by the subsequent axial introduction of each dilator segment or segments in order to achieve the final desired diameter for the access lumen.

The dilator probe itself forms the first dilation step in the stoma after puncturing of the target site. It terminates at a proximal end in a handle and at a distal end in a tapered cannula. The dilator probe generally takes the shape of a cannula having an annular cross section. However, over a specific portion of the cannula's length, opposing cylindrical sectors have been eliminated, resulting in two opposing arced lengths. These opposing arced lengths form slidable rails which engage with and allow longitudinal sliding of the plunger with respect to the dilator probe along a central longitudinal axis through the two components.

The plunger also terminates at a proximal end in a handle which in many embodiments may be similar to that of the dilator probe. The plunger generally takes the shape of a cannula and terminates at a distal end in an open lumen. Proximal to the distal end is a ridge or catch designed to engage at least some of the dilator segments. The plunger also contains an opposing arced configuration similar to that of the dilator probe. The arced lengths enable one of the two components to be axially rotated ninety (90°) degrees about its longitudinal axis with respect to the other which enables the two opposing arced lengths to slidably engage. The combination of the two components effectively creates a single slidable lumen through the central axis of the two components through which the penetration device may be inserted.

At least one, and often a plurality of dilator segments are provided. Each dilator segment is concentrically stacked over the cannulae portions of the dilator probe/plunger combination and one another should more than one dilator segment be provided. The dilator segments generally take the shape of cannulae as well. A distal end of each is gradually tapered whereas a proximal end may often be tapered in a much more aggressive manner. The distal end or ends are tapered to facilitate advancement of the member through the patient's anatomy, whereas the taper at the proximal end serves a different function. Specifically, the cannula sidewalls at the taper at the proximal end may be made to be rigid and resist buckling when subject to a compressive force. Rather than buckle under a compressive load, the dilator segment would move in the direction in which the force was applied. However, the side walls of the taper in the proximal end should allow passage of a member having a slightly larger diameter than the exit diameter of the lumen at the proximal end. The tapered sidewalls of the lumen should flex outward in a manner similar to a wiper seal upon passage of a slightly diametrically larger member.

Each dilator segment will have a length which is generally less than that of the dilator probe/plunger combination, and will have an outer diameter which is larger than the diameter of the combination. As stated, in many cases, the procedure to be performed will employ two or more dilator segments having successively larger diameters to provide for an incremental expansion, however, in some procedures the diameter of a single dilator segment will be sufficient to radially expand the stoma to its final desired diameter. Typically, the outer diameter of the largest dilator segment will be at least two fold larger than the diameter of the dilator probe/plunger combination, usually being at least three fold larger, and frequently being five fold or more larger. To assist in the longitudinal sliding motion of each of the components as well as to further facilitate penetration, the exterior surface of any of the components may be wholly or partly coated with a lubricant or be manufactured from a lubricious polymer such as polyethylene.

An optional splittable outer sheath may be provided and would serve functionally as the outermost concentrically disposed dilator segment. The outer sheath, if provided, should be detachable from the apparatus itself and therefore would be designed to remain in situ within the stoma. A suitable apparatus such as a surgical instrument or catheter could then be passed through the lumen of the outer sheath and into the patient. The outer sheath would then be split longitudinally and removed without requiring retraction or removal of the surgical instrument or catheter.

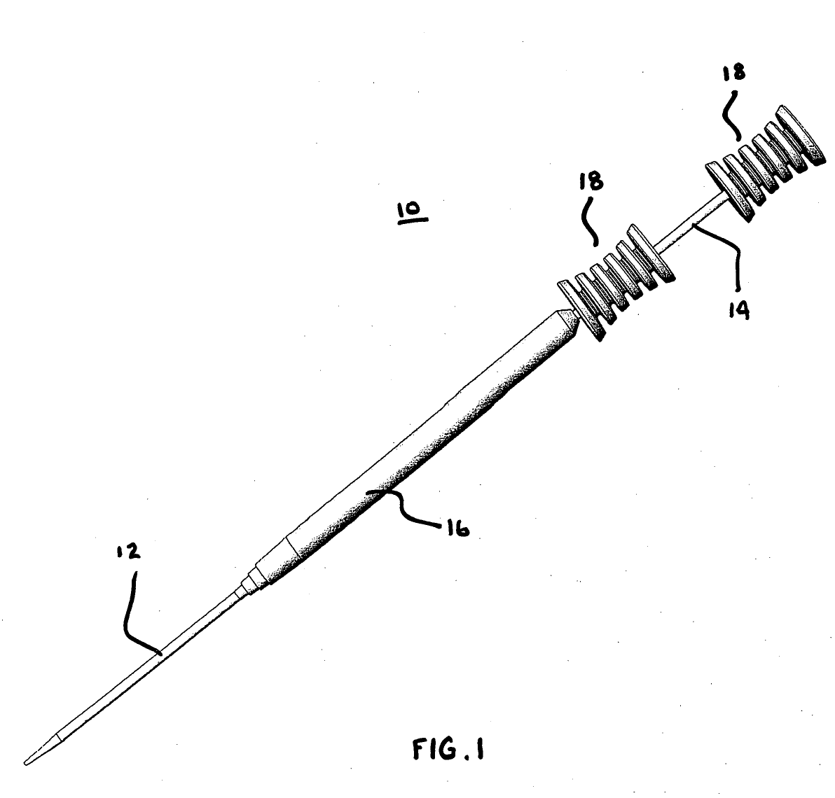

Referring now to FIG. 1, a percutaneous dilation apparatus or tool 10 constructed in accordance with the principles of the present invention is described in a first exemplary embodiment as a gastrointestinal tube placement tool. The apparatus 10 includes a dilator probe 12, a plunger 14, and at least one dilator segment 16. A handle 18 is provided on each, the dilator probe 12 and the plunger 14. Though the handles 18 are identically shaped in the FIGs., this is not meant to signify a limitation to the invention. In fact, it may be desirable to have different configurations for each handle to enable easier gripping, easier manipulation, or other desirable features or functions that would be understood by those of skill in the art.

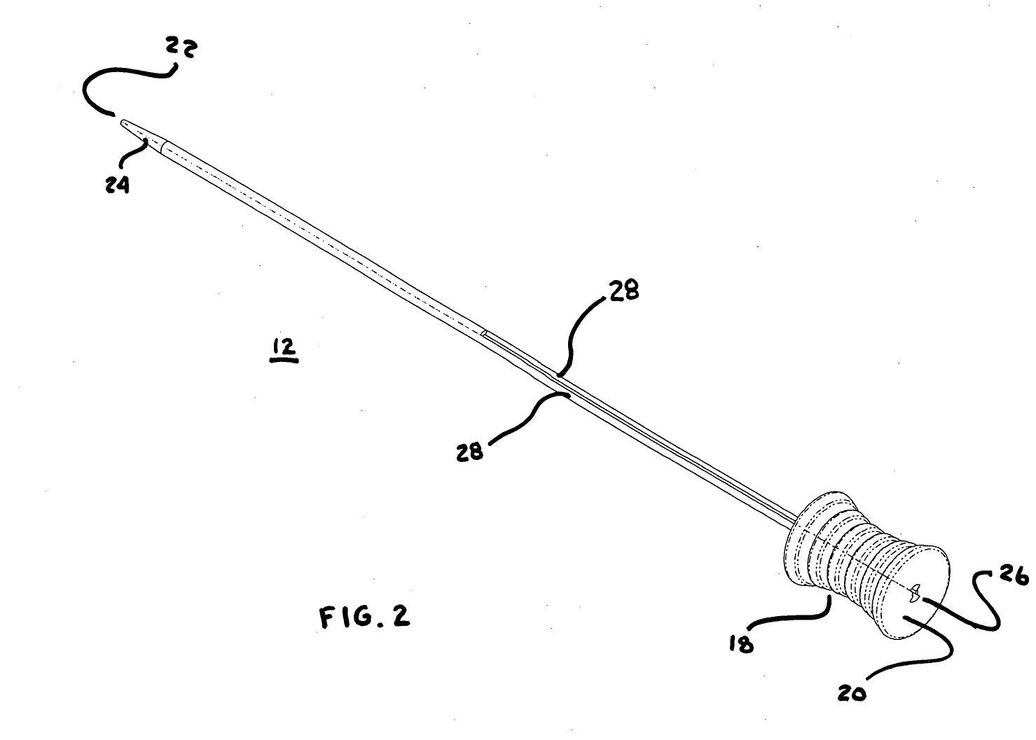

Looking now to FIG. 2, the dilator probe 12 is shown separated from the remaining components of the device 10. A proximal end 20, of the dilator probe 12 may be configured as the handle 18, previously described. A distal end 22 may be configured into the shape of a gradual taper 24. The taper 24 is designed to facilitate advancement of the tool 10 through a patient's anatomy. The dilator probe 12 generally takes the shape of a cannula having a lumen 26 that extends through a central longitudinal axis of the dilator probe 12. However, as may be seen in the FIG., over a specific portion of the probe 12, opposing cylindrical sectors have been removed from the probe 12. The resultant structure, in this embodiment, forms two opposing quarter-annular arcuate lengths 28 which serve as rails to slidably guide the plunger 14.

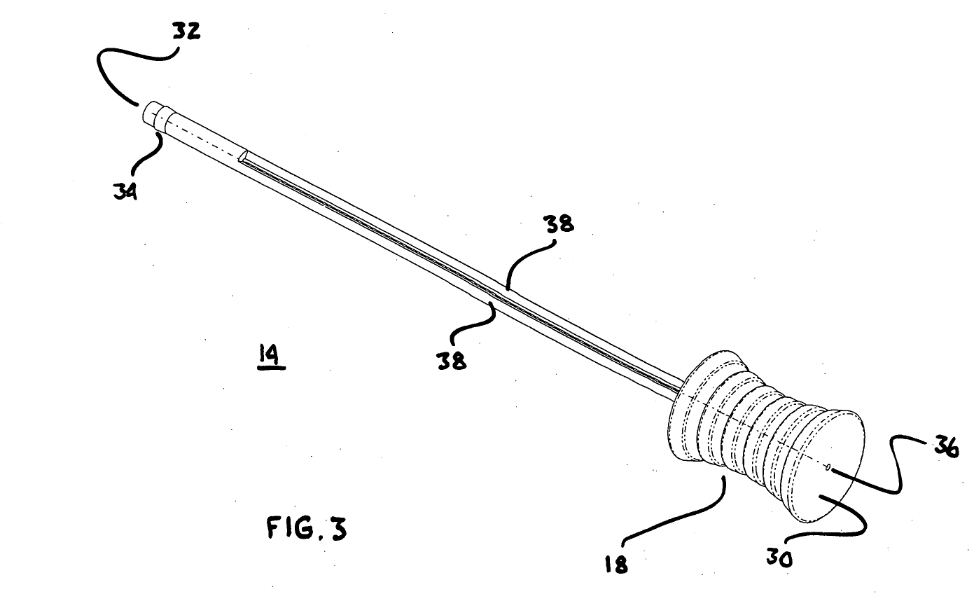

FIG. 3 depicts the plunger 14 separated from the remaining components of the device 10. A proximal end 30 of the plunger 14 may also be configured as a handle 18. A distal end 32 is also provided. Proximal to the distal end 32 is a catch 34, which in the illustrated embodiment is in the shape of a frustoconical cylinder. The plunger 14 forms a cannula having a lumen 36 that extends through a central longitudinal axis of the plunger 14. The plunger 14 also has a cross-sectional arrangement similar to that of the dilator probe 12 in that two opposing quarter-annular arcuate lengths 38 are provided to engage those of the dilator probe 12.

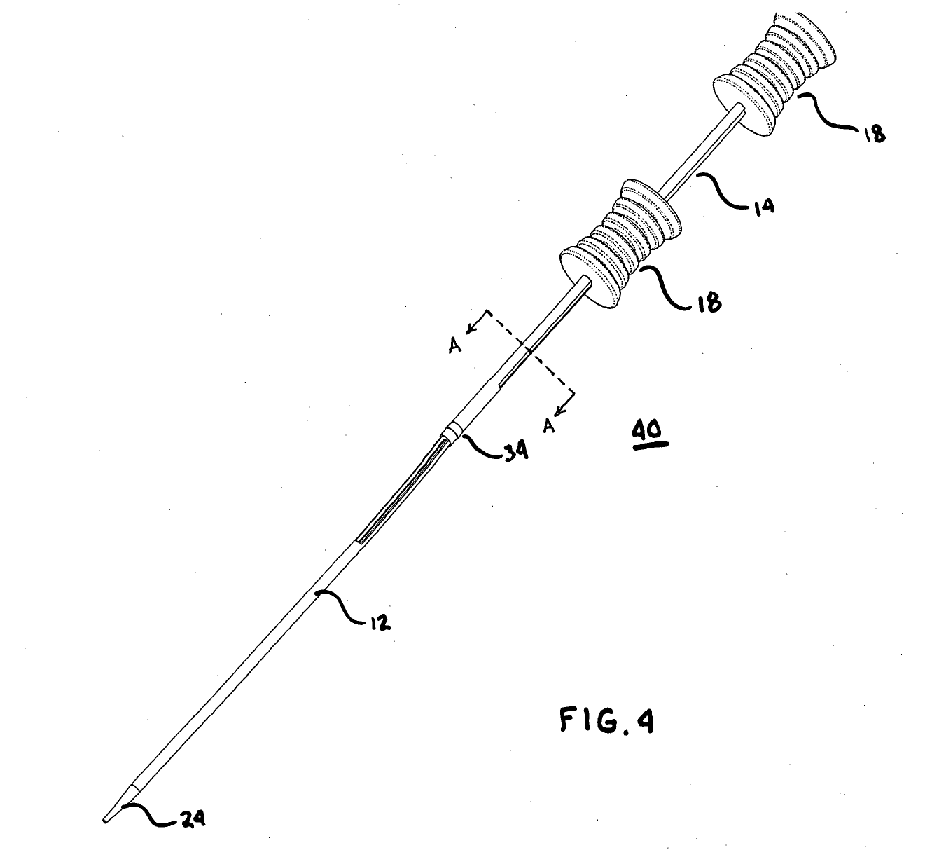

The two components, the dilator probe 12 and the plunger 14 when assembled form a slidable subassembly 40 as depicted in FIG. 4. As may be seen in the FIG., the plunger 14 is engaged with the dilator probe 12 and is slidable between two end positions. Moreover, the cross-sectional shape of the lumen 26 through the handle 18 on the dilator probe 12 is configured to accept the two opposing quarter-annular arcuate lengths as may be seen in FIG. 2. Of course, in the embodiment depicted, during manufacture the components would be engaged before the handles 18 were secured. This, however, would be understood by those of skill in the art. In other embodiments, the plunger may be made to be removable after performing its intended function as described below.

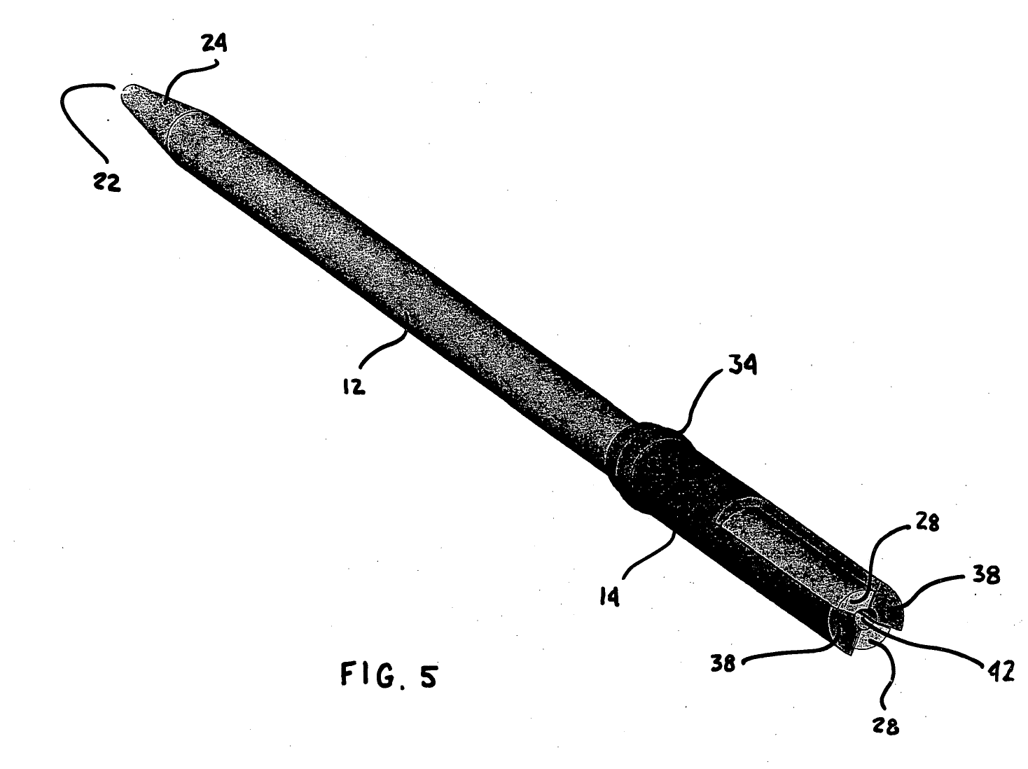

FIG. 5 depicts a cross-sectional view of the subassembly taken through the line A-A from FIG. 4. This view clearly depicts the opposing quarter-annular arcuate lengths 28 of the dilator probe 12 as well as the corresponding opposing quarter-annular arcuate lengths 38 of the plunger 14. From this view, one of skill in the art would understand how the components slide with respect to one another and how the two together jointly form a lumen 42 that combines portions of lumens 26 and 36 and is otherwise coaxially aligned. As briefly stated above, either or both of these components may be made lubricious or manufactured of lubricious polymers to decrease friction between them.

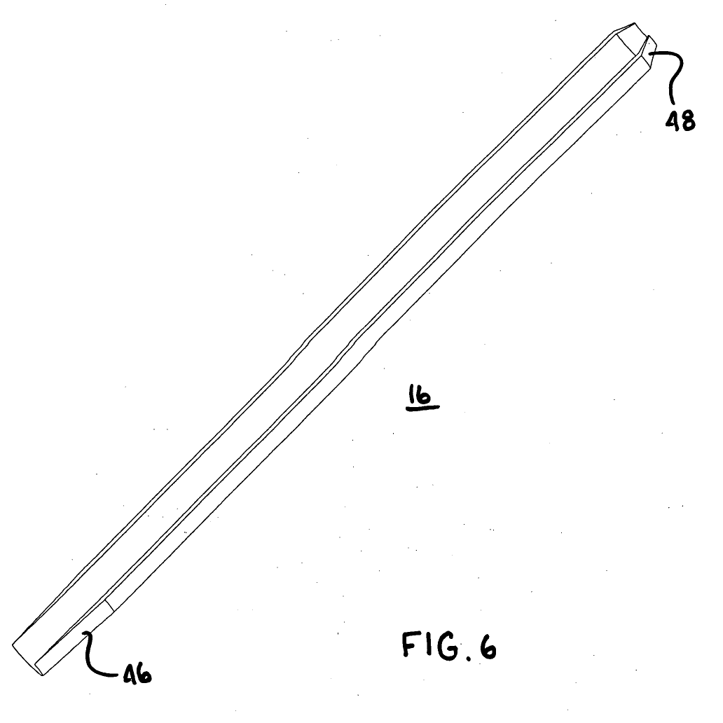

FIG. 6 is a cutaway view of a representative dilator segment 16. Each dilator segment 16 is generally a cannula. A distal end 44 of each is gradually tapered into, for example, straight taper 46 whereas a proximal end is often tapered as well into, for example, hollow taper 48. The straight taper 46 is designed to facilitate advancement of the apparatus 10 through the patient's anatomy and to stretch or dilate the stoma as it enters the patient. As such, the specific shape of the taper 46 is not crucial; however, it should be capable of gradual dilation of the stoma site as it is advanced. The length between the two tapers 46 and 48 is generally cylindrical in shape to enable easier sliding between other dilator segments or between the dilator segment and the dilator probe 12. The hollow taper 48 is designed to rapidly transition from the wall thickness of the cylindrical portion to a tip having a certain desired degree of flexibility as explained below. Specifically, the sidewall of the dilator segment 16 at the hollow taper 48 is designed to be rigid and resist buckling when subjected to a compressive force. Rather than buckle under a compressive load, the dilator segment 16 would be urged to move in the direction in which the force was applied.

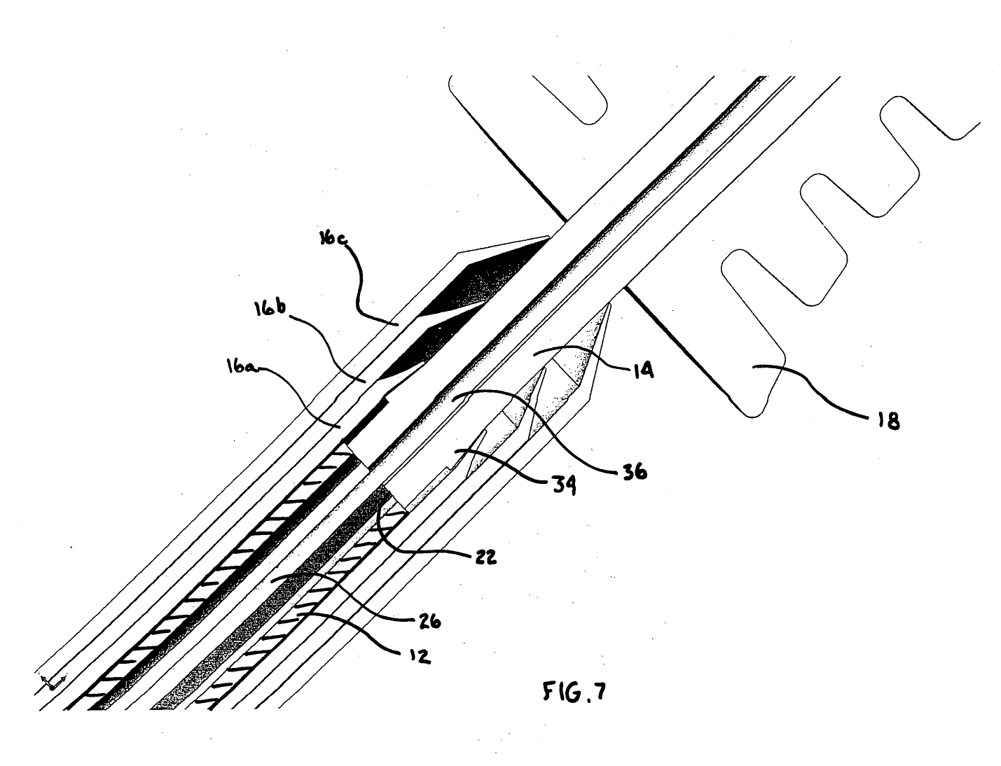

FIGS. 7, 8, and 9 depict enlarged cross-sectional cutaways of the apparatus 10 in various states of engagement. The handle 18 associated with the dilator probe 12 is depicted in FIG. 7. A plurality of dilator segments 16 are depicted and are labeled 16a, 16b, 16c for ease of description. In this case three are shown; however, the number of dilator segments is not meant to serve as a limitation to the invention. Regardless of the number, as is evident from this FIG., each dilator segment is disposed in a stacked or nested and concentrically oriented arrangement about the dilator probe 12. As such, each successively larger diameter dilator segment envelops the next segment. Each dilator segment is capable of sliding along its central axis and may be made lubricious or manufactured of lubricious polymers to decrease friction between them as well.

For clarity, the illustration of FIG. 7 may not be to scale, but it is meant to show the relative movement of the components, not necessarily their actual placement along the longitudinal axis of the tool itself. That being said, it may be seen that the plunger 14 is in contact at its distal end 22 with that portion of the dilator probe 12 that is annular in cross section. The catch 34 is within the central bore or lumen of the dilator segment 16a, so as would be understood, the dilator segment is concentrically oriented about an exterior of the dilator probe and a portion of the plunger. This position may be thought of as an initial position in which none of the dilator segments 16 are extended distally toward a distal end of the tool 10. Were the device to actually be in this position, in looking at FIG. 1, both handles 18 would be in physical contact (rather than separated as FIG. 1 actually depicts) and the dilator segments 16a, 16b, and 16c would be located proximal to the handle 18 of the dilator probe 12 as shown.

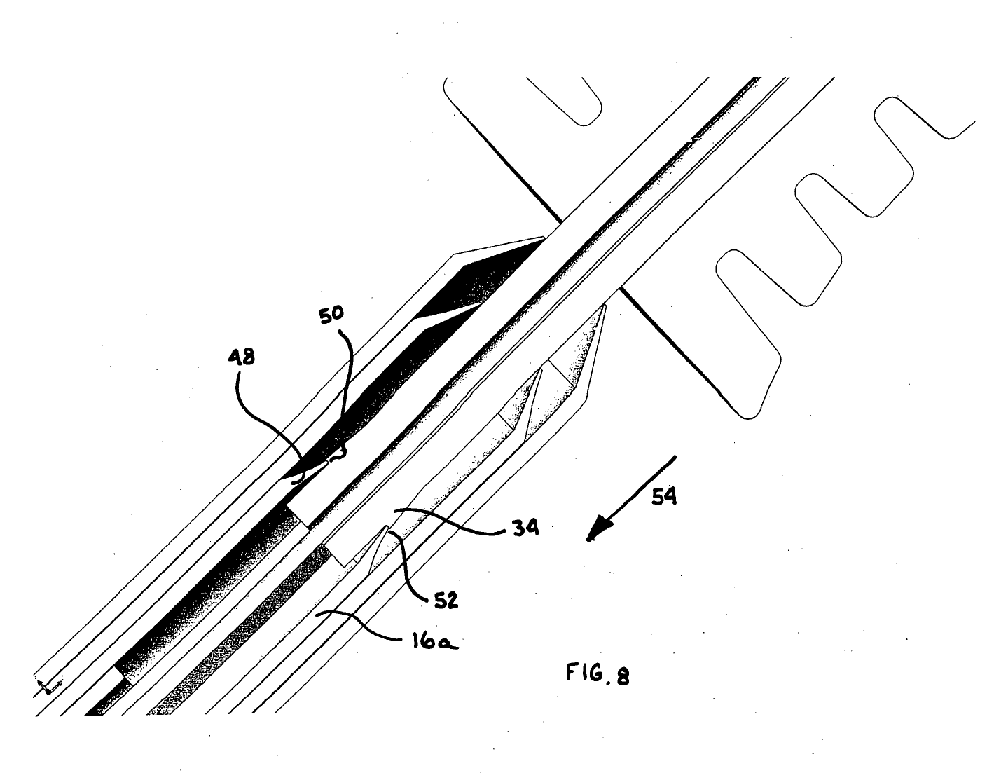

Pulling back on the plunger 14 so that the handles 18 are separated from one another causes the plunger 14 to be removed and no longer concentrically disposed within the dilator segment 16a. Due to the construction of the hollow taper 48, and the shape of the catch 34, the sidewalls of the segment 16a at the taper 48 would flex outward in a manner similar to a wiper seal thus allowing relatively effortless passage and disengagement of the catch 34. However, now turning to FIG. 8, it may be seen that were the plunger 14 to be moved such that its handle 18 is pushed toward the handle 18 of the dilator probe 12, a flange 50 located on a distal facing edge of the catch 34 will contact a proximally facing end 52 on the dilator segment 16a. Since the hollow taper 48 is designed to be rigid and resist buckling under application of this compressive force, rather than buckle, the dilator segment 16a is urged to move distally in the direction of arrow 54 or toward the distal end of the apparatus 10.

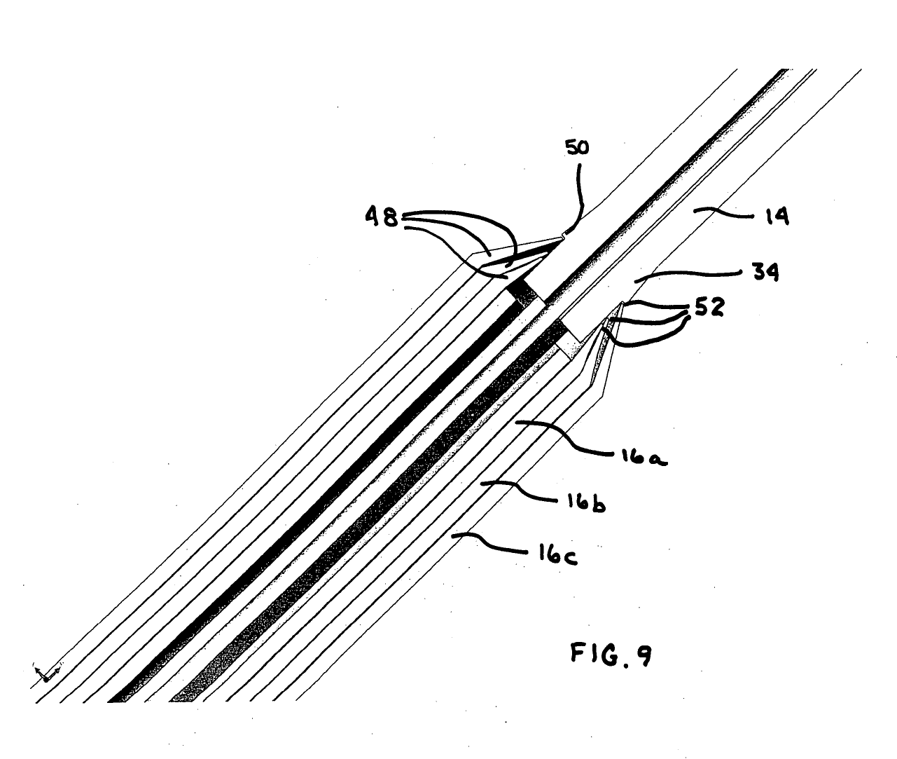

This process is repeatable and results in the sequential movement of each of the dilator segments 16a, 16b, and 16c in turn. FIG. 9 depicts a point in time wherein each of the plurality of dilator segments has been moved distally along the dilator probe 12. As may be seen each of the dilator segments has a hollow taper 48 that is formed in such a manner so as to enable each of the facing ends 52 of each dilator segment 16a, 16b, and 16c to engage the flange 50 on the catch 34. The configuration described is meant to enable the plunger 14 to have the ability to manipulate the dilator segments 16 in turn longitudinally along the length of the dilator probe 12 and other possible arrangements are contemplated so long as they function in accordance with the description in that each dilator segment is moved sequentially in turn.

Now that the components and their operation have been described, it would be understood by those having skill in the art that in many cases, the procedure to be performed will employ two or more dilator segments having successively larger diameters to provide for an incremental expansion. However, in some procedures the diameter of a single dilator segment will be sufficient to radially expand the stoma to its final desired diameter. Typically, the outer diameter of the largest dilator segment will be at least two fold larger than the diameter of the dilator probe/plunger combination, usually being at least three fold larger, and frequently being five fold or more larger.

In many of the embodiments, the dilator segments are meant to be moved individually and in sequence. As such, indication of engagement for each dilator segment may be desirable. For example indicia may be applied to the plunger to indicate how far to manipulate it in order to engage each dilator segment in turn. An alternative embodiment may rely upon an audible click or other sound that would indicate that the catch had disengaged from within the dilator segment to next be moved. Other solutions could include the placement of a detent or other mechanism to indicate that the next dilator segment is capable of placement.

In another embodiment, an outer sheath may be provided. The outer sheath could simply be an outermost concentrically oriented dilator segment. However, the sheath would be removable from the remainder of the apparatus. An advantage to this construction is that the apparatus 10 may be placed in the target location within the patient's body, the outer sheath advanced into the stoma by detaching it from the handle from a suitable connector such as a luer fitting or other threaded connector and moving the sheath into the penetration. Once the sheath is disposed within the penetration site, the apparatus may be removed without disturbing the sheath. This configuration allows for the sheath to maintain the stoma in a fully dilated condition thus enabling the subsequent insertion of a medical instrument. The sheath may be splittable longitudinally along one or more weakened lines enabling it to be split, for example, into two halves and subsequently removed without disturbing the medical instrument placed within the patient through the stoma.

As used herein and in the claims, the term “comprising” is inclusive or open-ended and does not exclude additional unrecited elements, compositional components, or method steps.

While various patents may have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A percutaneous dilation apparatus comprising:
a dilator probe for entering and enlarging a percutaneous penetration into a target location within a patient's body, the dilator probe having a handle at a proximal end, a tapered distal end, and defining a length therebetween;
a plunger having a handle at a proximal end, a distal end, a catch proximal to the distal end, and a length defined between the proximal and distal ends wherein the plunger slidably engages the dilator probe;
at least one dilator segment having a tapered distal end, the segment disposed in an initial configuration concentrically about an exterior of the dilator probe and a portion of the plunger and upon withdrawal of the plunger from the dilator segment and subsequent application of force to the dilator segment by the plunger, the catch moves the at least one dilator segment along the length of the dilator probe to a final position proximate the distal end of the dilator probe wherein the tapered distal end of the dilator segment enters and enlarges the percutaneous penetration an amount greater than the dilator probe alone.

2. The apparatus of claim 1 comprising a plurality of concentrically nested dilator segments.

3. The apparatus of claim 1 wherein the dilator probe and plunger are arranged to form a central lumen through a longitudinal central axis of the combination.

4. The apparatus of claim 1 comprising a penetration device.

5. The apparatus of claim 1 comprising a plurality of cylindrical dilator segments graduated in size from smaller to larger, and concentrically arranged about the dilator probe and the next smaller dilator segment, each segment being engaged sequentially by the catch and moved toward the distal end to enable enlargement of the penetration.

6. A percutaneous dilation apparatus comprising:
a movable dilator segment having a tapered distal end adapted to enter and expand a percutaneous penetration in a target location within a patient's body;
a dilator probe having a tapered distal end for initial entry and initial enlargement of the percutaneous penetration, the dilator probe disposed internally and concentrically within the movable dilator segment, and having a mechanism for releasably engaging the movable dilator segment and moving it from an initial position to a deployed position within the percutaneous penetration.

7. The apparatus of claim 6 comprising a plurality of cylindrical dilator segments graduated in size from smaller to larger, and concentrically arranged about the dilator probe and the next smaller dilator segment, each segment being engaged sequentially by the catch and moved toward the distal end to enable enlargement of the penetration.

8. A percutaneous dilation apparatus comprising:
a plurality of dilator segments, diametrically graduated and concentrically nested one within the other;
a dilator probe for initial entry and enlargement of a percutaneous penetration in a target location within a patient's body, the dilator probe having a mechanism for releasably engaging each movable dilator segment sequentially from smallest to largest and moving each movable dilator segment in turn along the dilator probe and into the penetration thereby dilating the penetration with the insertion of each dilator segment.

9. The apparatus of claim 8 comprising an outer sheath concentrically oriented around the outermost dilator segment, the sheath being removable from the remainder of the apparatus so that it stays in place within the penetration, the sheath adapted to have a medical instrument passed therethrough and into the patient's body, the sheath being longitudinally splittable so that it may be subsequently removed from the penetration without disturbing the medical instrument placed within the patient's body.
